(19) United States
(12) Patent Application Publication
(10) Pub. No.: US 2008/0153903 A1
(43) Pub. Date: Jun. 26, 2008

(54) INHIBITORS OF PROTEIN KINASE C-DELTA FOR THE TREATMENT OF GLAUCOMA

(75) Inventors: Debra L. FLEENOR, Crowley, TX (US); Iok-Hou PANG, Grand Prairie, TX (US); Allan R. SHEPARD, Fort Worth, TX (US); Abbot F. CLARK, Arlington, TX (US)

Correspondence Address: ALCON, IP LEGAL, TB4-8, 6201 SOUTH FREEWAY, FORT WORTH, TX, 76134, US

(73) Assignee: ALCON MANUFACTURING, LTD., Fort Worth, TX (US)

(21) Appl. No.: 11/961,493

(22) Filed: Dec. 20, 2007

Related U.S. Application Data
(60) Provisional application No. 60/871,524, filed on Dec. 22, 2006.

(51) Int. Cl. A61K 31/352; A61P 27/02

(52) U.S. Cl. 514/456

(57) ABSTRACT

The present invention relates to methods to treat and/or prevent increased intraocular pressure in a subject by administering a protein kinase C-delta (PKCδ) inhibitor. In further embodiments, the present invention relates to methods to treat and/or prevent glaucoma by administering a PKCδ inhibitor.

### CROSS-REFERENCE TO RELATED APPLICATION

This application claims priority under 35 U.S.C. §119 to U.S. Provisional Patent Application No. 60/871,524 filed Dec. 22, 2006, the entire contents of which are incorporated herein by reference.

### TECHNICAL FIELD

The present invention relates to the use of compounds that inhibit protein kinase C-delta thereby treating and/or preventing glaucoma or increases in intraocular pressure.

### BACKGROUND OF THE INVENTION

### I. Glaucoma and Elevated Intraocular Pressure

Glaucomatous optic neuropathy (glaucoma) is a disease characterized by the permanent loss of visual function due to irreversible damage to the optic nerve. The several morphologically or functionally distinct types of glaucoma are typically characterized by elevated intraocular pressure (IOP), which is considered to be causally related to the pathological course of the disease. Examples include primary open angle glaucoma (POAG) and angle closure glaucoma.

Drug therapies that have proven to be effective for the reduction of IOP and/or the treatment of POAG include both agents that decrease aqueous humor production and agents that increase the outflow facility. Such therapies are in general administered by one of two possible routes; topically (direct application to the eye) or orally. However, pharmaceutical ocular anti-hypertension approaches have exhibited various undesirable side effects. For example, miotics such as pilocarpine can cause blurring of vision, headaches, and other side effects. Systemically administered carbonic anhydrase inhibitors can also cause nausea, dyspepsia, fatigue, and metabolic acidosis. Certain prostaglandins cause hyperemia, ocular itching, and darkening of eyelashes and periorbital skin. Such negative side-effects may lead to decreased patient compliance or to termination of therapy such that vision continues to deteriorate. Additionally, there are individuals who simply do not respond well when treated with existing glaucoma therapies. There is, therefore, a need for other therapeutic agents for the treatment of ocular disorders such as glaucoma and elevated IOP or ocular hypertension.

### II. Protein Kinase C

Protein kinase C-delta (PKCδ) is a member of the protein kinase C family of enzymes that includes at least eleven serine/threonine kinase isozymes. Of these, the isozymes can be classified into three different subgroups based on calcium-dependency and whether their activation is triggered by diacylglycerol. These subgroups include: (1) “conventional” (e.g., α, β1, β2, γ) (2) “atypical” (e.g., ζ, λ), and (3) “novel” (δ, ε, η, θ, μ). PKCδ affects multiple systems which, ultimately, may contribute to the pathogenesis of glaucoma. For example, it has been implicated as playing a role in the induction of glucose-induced fibronection expression (Mueller et al., 1997) and upregulation of collagen gene and protein expression. (Jinnin et al., 2005; Zhang et al., 2004; and Runyan et al., 2003). Both collagen and fibronection are components of the extracellular matrix, and an overaccumulation has been proposed as contributing to the increased aqueous humor outflow resistance/elevation of intraocular pressures such as seen in POAG. PKCδ has also been shown to activate Rho kinase in porcine coronary arteries (Kandabashi et al., 2003). Rho kinase has been postulated to be an important modulatory agent for the outflow of aqueous humor (Rao et al, 2005).

PKCδ has also been shown to be crucial not only for transforming growth factor (TGFβ)-inducted fibronection synthesis, but also for expression of the Smad3 transcription factor protein. Various groups have reported significantly-higher levels of a TGFβ2 isoform in aqueous humor (AH) collected from glaucomatous human eyes, as compared to “normal” eyes. Furthermore, it has been demonstrated that TGFβ2 can provoke substantial increases in intraocular pressure.

Thus, the present invention is the first to demonstrate the use of inhibitors of PKCδ as a means to treat and/or prevent glaucoma or ocular hypertension.

### BRIEF SUMMARY OF THE INVENTION

The present invention is directed to treating and/or preventing increases in intraocular pressure in a subject and/or glaucoma.

One embodiment of the present invention comprises a method of decreasing intraocular pressure in an eye of a subject comprising administering to the subject an effective amount of a composition comprising a protein kinase C-delta (PKCδ) inhibitor.

The PKCδ inhibitor can include a small molecule, for example, but not limited to bisindolylmaleimide I, bisindolylmaleimide II, bisindolylmaleimide III, bisindolylmaleimide IV, calphostin C, chelerythrine chloride, ellagic acid, Gö 7874, Gö 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, D-erythro-sphingosine, and any analog thereof or structural equivalent thereof and/or mimetic thereof. In specific embodiments, the PKCδ inhibitor is rottlerin.

The subject of the present invention can be a subject that is susceptible to having elevated intraocular pressure, susceptible to having glaucoma or is at risk for developing glaucoma, has ocular hypertension, and/or has glaucoma, for example open angle glaucoma. In some embodiments, elevated intraocular pressure is defined as intraocular pressure in an eye of the subject that is 21 mm Hg or greater. In some embodiments, the intraocular pressure is less than 21 mm Hg, but the subject is a subject that is known to be at risk of developing glaucoma. For example, the subject may have a family history of glaucoma. In some embodiments, the subject has findings on examination consistent with a diagnosis of glaucoma but yet does not have elevated intraocular pressure (i.e., normotensive glaucoma).

In further embodiments, the PKCδ inhibitor can be administered in combination with a known agent to treat glaucoma and/or ocular hypertension. Such agents can include, but are not limited to β-blockers, prostaglandin analogs, carbonic anhydrase inhibitors, α2 agonists, miotics, neuroprotectants, rho kinase inhibitors, and combinations thereof.

Another embodiment of the present invention comprises a method of treating and/or preventing glaucoma comprising administering to a subject having glaucoma or a subject susceptible to having glaucoma an effective amount of a composition comprising a protein kinase C-delta inhibitor (PKCδ).

Another embodiment of the present invention comprises a method of decreasing transforming growth factor beta-2 (TGFβ2) activity in ocular tissue comprising administering to a subject suspected of having increased intraocular pressure an effective amount of a protein kinase C-delta inhibitor (PKCδ) in an amount sufficient to decrease TGFβ2 activity. A decrease in TGFβ2 activity results in a decrease in intraocular pressure, which in turn results in treatment or prevention of glaucoma.

Yet further, another embodiment comprises a method of manufacturing a protein kinase C-delta (PKCδ) inhibitor comprising: (a) providing a candidate substance suspected of decreasing PKCδ activity; (b) selecting the PKCδ inhibitor by assessing the ability of the candidate substance to decrease PKCδ; and (c) manufacturing the selected PKCδ inhibitor. The candidate substance is a small molecule. Once the PKCδ inhibitor is identified, it can be used to treat/prevent glaucoma and/or ocular hypertension or elevated intraocular pressure.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. Figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawing and wherein:

FIG. 1A, FIG. 1B and FIG. 1C depict the effects of various amounts of PKC inhibitors on basal and stimulated (TGFβ2) fibronectin content in supernatants collected from treated monolayers of GTM-3 cells. FIG. 1A shows that the specific PKCδ inhibitor rottlerin effectively reduces TGFβ2-stimulated fibronectin levels at both tested concentrations; rottlerin also effectively reduced basal fibronectin at the 10 uM dose. FIG. 1B demonstrates that Go6976, a PKC inhibitor which does not inhibit the PKCδ isozyme, was without effect on either basal or TGFβ2-stimulated fibronectin content. FIG. 1C depicts the lack of effect of the broad-spectrum PKC inhibitor Bisindolylmaleimide I (Bis I) on fibronectin levels; at the tested concentrations, Bis I might have only a weak inhibitory effect on PKCδ. Thus, these data demonstrate that PKCδ activity is essential to fibronectin production and/or release by TM cells.

FIG. 2A, FIG. 2B and FIG. 2C depicts the effects of PKC inhibition on basal and stimulated (TGFβ2) PAI-I content in supernatants collected from treated monolayers of GTM-3 cells. FIG. 2A shows that the specific PKCδ inhibitor rottlerin dose-dependently reduces TGFβ2-stimulated PAI-I levels. FIG. 2B demonstrates that Gö6976 was without effect on either basal or TGFβ2-stimulated PAI-I content. FIG. 1C shows that Bis I reduced TGFβ2-stimulated PAI-I levels only at the 100 nM dose, a concentration at which Bis I can exhibit minor inhibitory effects on PKC-delta. Thus, these data demonstrate that PKCδ activity is essential to PAI-I production and/or release by TM cells.

FIG. 3 shows the effect of single concentrations of the same PKC inhibitors on the levels of pro-collagen Type I C-peptide (PIP) in supernatants from treated GTM-3 cell monolayers. Only rottlerin elicited a significant effect in this model, indicating the essential role of PKCδ in the GTM-3 cellular response.

### DESCRIPTION OF ILLUSTRATIVE EMBODIMENTS

### I. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

As used herein, the term “analogs” may include structural equivalents or mimetics.

As used herein, the term “effective amount” refers to an amount of the agent that will decrease or reduce or inhibit the function and/or activity of protein kinase C-delta (PKCδ). The reduction of PKCδ results in a decrease of Smad3 expression and/or activity, reduction in transforming growth factor-beta (TGFβ2) expression and/or activity, reduction in intraocular pressure, etc. Thus, an effective amount is an amount sufficient to detectably and repeatedly ameliorate, reduce, minimize or limit the extent of the disease or its symptoms.

As used herein, the term “intraocular pressure” or “IOP” refers to the pressure of the fluid inside the eye. In a normal human eye, IOP is typically in the range of 10 to 21 mm Hg. IOP varies among individuals, for example, it may become elevated due to anatomical problems, inflammation of the eye, as a side-effect from medication or due to genetic factors. “Elevated” intraocular pressure is usually considered to be >21 mm Hg, which is also considered to be a risk factor for the development of glaucoma. However, some individuals with an elevated IOP may not develop glaucoma and are considered to have ocular hypertension.

As used herein, the terms “glaucomatous optic neuropathy” or “glaucoma” are interchangeable. Glaucoma refers to a disease characterized by the permanent loss of visual function due to irreversible damage to the optic nerve. The two main types of glaucoma are primary open angle glaucoma (POAG) and angle closure glaucoma.

As used herein, the term “inhibitor” refers to a molecule or compound that acts to suppress the expression or function of another biological substance. More specifically, the “inhibitor” decreases the biological activity of a gene, an oligonucleotide, protein, enzyme, signal transducer, receptor, transcription activator, co-factor, and the like. Such inhibition may be contingent upon occurrence of a specific event, such as activation of a signal transduction pathway and/or may be manifest only in particular cell types. In specific embodiments, the inhibitor decreases PKCδ signaling pathways resulting in a decrease in TGFβ-mediated responses, a decrease in Smad3 activity and/or expression, etc.

As used herein, the terms “susceptible,” or “susceptibility” refers to an individual or subject that is or at risk of developing glaucoma. For example, the subject may have elevated intraocular pressure in one or both eyes without any other findings associated with glaucoma. While such an individual does not clinically carry a diagnosis of glaucoma, such an individual is at risk of developing glaucoma by virtue of the presence of the elevation in intraocular pressure. For example, the intraocular pressure may be greater or equal to 21 mm Hg in one or both eyes. A subject without elevated intraocular pressure who does not have glaucoma may also be susceptible to the development of glaucoma. For example, the subject may have a family history of glaucoma. The subject may or may not have a family history of glaucoma. “Susceptibility” is determined and assessed by any method known to those of ordinary skill in the art. For example, susceptibility can be determined based on results of physical examination, family history, or genetic screening techniques well-known to those of ordinary skill in the art.

As used herein, the term small molecule can include, nucleic acids, proteins, peptides, polypeptides, etc.

As used herein, the term “patient” or “subject” refers to a mammal. Preferred patients and subjects are humans.

As used herein, the terms “treatment” and “treating” refer to administration or application of a therapeutic agent to a subject or performance of a procedure or modality on a subject for the purpose of obtaining a therapeutic benefit of a disease or health-related condition. Thus, one of skill in the art realizes that a treatment may improve the disease condition, but may not be a complete cure for the disease. Treatment also includes reducing the risk of developing more severe disease in a subject with a disease.

As used herein, the term “therapeutic benefit” or “therapeutically effective” as used throughout this application refers to anything that promotes or enhances the well-being of the subject with respect to the medical treatment of his condition. This includes, but is not limited to, a reduction in the frequency or severity of the signs or symptoms of a disease. Therapeutic benefit also includes a reduction in intraocular pressure compared to intraocular pressure in the absence of the therapeutic agent. Therapeutic benefit also includes reducing the signs or symptoms associated with glaucoma in a subject with glaucoma. For example, a therapeutic benefit in a patient with glaucoma is obtained where there is no further progression of visual field loss in the affected eye, or a slowing of the rate of progression of visual field loss in the affected eye.

As used herein, the terms “prevention” and “preventing” as used herein are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition. An individual with an eye that is at risk of developing glaucoma can be treated with a PKCδ inhibitor as set forth herein for the purpose of blocking the onset of the signs or symptoms of glaucoma (i.e., prevention of glaucoma).

### II. Treatment/Prevention

In certain aspects of the present invention, compounds are used to treat and/or prevent glaucoma and/or elevated intraocular pressure. More particularly, the compounds are used to inhibit, reduce or block PKCδ enzyme activity. Reduction and/or inhibition of PKCδ activity will delay the progression of the disease, decrease intraocular pressure, decrease TGFβ2 activity, which alters or reduces TFGP2-induced fibronectin synthesis, decrease Smad3 protein activity and/or gene expression, etc.

A subject to be treated using the PKCδ inhibitors can be a subject suffering from glaucoma or one that has elevated intraocular pressure or ocular hypertension. Other subjects that can be treated using the PKCδ inhibitors can be a subject who is known or suspected of being free of a glaucoma or related condition at the time the PKCδ inhibitor is administered. The subject, for example, can be a subject with no known disease or health-related condition (i.e., a healthy subject). In some embodiments, the subject is a subject at risk of developing glaucoma or at risk for developing elevated intraocular pressures. Thus, in certain embodiments of the invention, methods include identifying a patient in need of treatment. A patient may be identified, for example, based on taking a patient history, or based on findings on clinical examination. For example, a subject at risk for elevated intraocular pressure or ocular hypertension can be a subject having an intraocular pressure of greater than 21 mm Hg. A subject may be any vertebrate, such as a mammal. In particular embodiments, the subject is a human.

PKCδ inhibitors that can be used in the present invention include small molecules. Examples of small molecules that may be screened include, but are not limited to, small organic molecules, peptides or peptide-like molecules, nucleic acids, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that inhibit the activation PKCδ. Further, in drug discovery, for example, proteins have been fused with antibody Fc portions for the purpose of high-throughput screening assays to identify potential modulators of new polypeptide targets. See, D. Bennett et al., Journal of Molecular Recognition, 8: 52-58 (1995) and K. Johanson et al., The Journal of Biological Chemistry, 270, (16): 9459-9471 (1995).

More specifically, the small molecule can include, but is not limited to the following compounds bisindolylmaleimide I, bisindolylmaleimide II, bisindolylmaleimide III, bisindolylmaleimide IV, calphostin C, chelerythrine chloride, ellagic Acid, Gö 7874, Gö 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, and D-erythro-sphingosine.

Yet further, PKCδ inhibitors can include structural equivalents or mimetics that can be generated using techniques of modeling and chemical design known to those of skill in the art. The art of computer-based chemical modeling is now well known. Using such methods, a chemical that specifically inhibits PKCδ can be designed, and then synthesized, following the initial identification of a compound that inhibits PKCδ activity and/or induction, but that is not specific or sufficiently specific to inhibit PKCδ activity in individuals suffering from glaucoma and/or elevated intraocular pressure. It will be understood that all such sterically similar constructs and second generation molecules fall within the scope of the present invention.

Treatment and/or prevention methods will involve treating an individual with an effective amount of a composition containing a PKCδ inhibitor. An effective amount is described, generally, as that amount sufficient to detectably and repeatedly to ameliorate, reduce, minimize or limit the extent of a disease or its symptoms. In the context of prevention, an effective amount is generally an amount that is sufficient to block the onset of a disease or its symptoms. More specifically, it is envisioned that the treatment with the PKCδ inhibitor thereof will stabilize or improve visual function (as measured by visual acuity, visual field, or other method known to those of ordinary skill in the art), decrease intraocular pressure, alter the PKC pathway, for example, decrease TGFβ2 activity, decrease Smad3 activity, etc. Thus, by administering the PKCδ inhibitor of the present invention, the PKC signaling pathway is disrupted resulting in downregulation of TGFβ2-mediated responses thereby affecting glaucoma's pathogenesis.

Furthermore, the compounds can be used to prevent the onset or delay the onset or reduce the severity of glaucoma and/or increased intraocular pressure. For example, a subject may not exhibit any clinical symptoms, but may have a family history or other risk factor for glaucoma and/or increased intraocular pressure. Thus, the PKCδ inhibitors of the present invention can prevent the onset, delay the onset or reduce the severity of glaucoma and/or increased intraocular pressure in the subject. Thus, treatment can include administering the PKCδ inhibitors to a subject at risk for developing glaucoma and/or at risk for developing intraocular hypertension.

### III. Combination Treatments

In order to increase the effectiveness of the methods of the present invention, it may be desirable to combine the PKCδ inhibitors with standard glaucoma treatments known and used by those of skill in the art.

The PKCδ inhibitors may precede or follow the additional agent treatment by intervals ranging from seconds to weeks to months. In other aspects, the PKCδ inhibitors may be administered simultaneously with the additional agent, for example, one composition containing both compounds or separate compositions can be administrated simultaneously.

A. Pharmaceutical Treatments

Examples of pharmacological agents to treat glaucoma that can be used in combination with the PKCδ inhibitors of the present invention include beta-blockers, such as timolol and betaxolol, and carbonic anhydrase inhibitors, such as dorzolamide and brinzolamide. Other agents may also include, prostaglandin analogs, which are believed to reduce intraocular pressure by increasing uveoscleral outflow, has become common. Three marketed prostaglandin analogs are latanoprost, bimatoprost and travoprost. Still further, other agents that may be used in combination with the PKCδ inhibitors may also include rho kinase inhibitors, α2 agonists, miotics, serotonergic agonists and neuroprotectants.

These pharmaceutical agents are typically administered topically, and work to either reduce aqueous production or they act to increase outflow.

B. Surgical Treatments

In addition to pharmacological agents, surgical procedures can be performed in combination with the administration of the PKCδ inhibitors. One such surgical procedure can include, laser trabeculoplasty. In laser trabeculoplasty, energy from a laser is applied to a number of noncontiguous spots in the trabecular meshwork. It is believed that the laser energy stimulates the metabolism of the trabecular cells, and changes the extracellular material in the trabecular meshwork.

Another surgical procedure may include filtering surgery. With filtering surgery, a hole is made in the sclera near the angle. This hole allows the aqueous fluid to leave the eye through an alternate route. The most commonly performed filtering procedure is a trabeculectomy. In a trabeculectomy, a conjunctiva incision is made, the conjunctiva being the transparent tissue that covers the sclera. The conjunctiva is moved aside, exposing the sclera at the limbus. A partial thickness scleral flap is made and dissected half-thickness into the cornea. The anterior chamber is entered beneath the scleral flap and a section of deep sclera and/or trabecular meshwork is excised. The scleral flap is loosely sewn back into place. The conjunctival incision is tightly closed. Post-operatively, the aqueous fluid passes through the hole, beneath the scleral flap which offers some resistance and collects in an elevated space beneath the conjunctiva called a bleb. The fluid then is either absorbed through blood vessels in the conjunctiva or traverses across the conjunctiva into the tear film.

### IV. Methods of Manufacturing PKCδ Inhibitors

The present invention contemplates methods for manufacturing inhibitors that affect the activity of PKCδ. These methods may comprise random screening of large libraries of candidate substances; alternatively, the methods may be used to focus on particular classes of compounds selected with an eye towards structural attributes that are believed to make them more likely to modulate the function or activity of PKCδ.

By function, it is meant that one may assay for protein activity, binding activity, etc. For example, a PKCδ inhibitor will be able to interfere with TGFβ2-mediated responses that result in glaucoma's pathogenesis. Thus, functional assays may include the measurement of TGFβ mediated responses that would be altered in view of inhibition of PKCδ. TGFβ2-mediated response can include, but are not limited to altered levels of fibronection, plasminogen activator inhibitor-1 (PAI-1), and pro-collagen I type C-peptide (PIP). Altered levels of fibronection, PAI-I and PIP may result from, for example, but not limited to decreases in gene expression, decreases in mRNA stability, decreases in protein synthesis, increases in protease activity and/or increases in protein degradation.

A. Inhibitors

The present invention further comprises methods for identifying, making, generating, providing, manufacturing or obtaining PKCδ inhibitors. PKCδ polypeptide may be used as a target in identifying compounds that decrease PKCδ activity. These assays may comprise random screening of large libraries of candidate substances; alternatively, the assays may be used to focus on particular classes of compounds selected with an eye towards structural attributes that are believed to make them more likely to inhibit the function of PKCδ.

To identify, make, generate, provide, manufacture or obtain a PKCδ inhibitor, one generally will determine the activity of PKCδ in the presence, absence, or both of the candidate substance, wherein an inhibitor is defined as any substance that decreases, reduces, abrogates or inhibits PKCδ activity. For example, a method may generally comprise:
(a) providing a candidate substance suspected of decreasing PKCδ activity;
(b) assessing the ability of the candidate substance to decreases PKCδ activity;
(c) selecting PKCδ inhibitor; and
(d) manufacturing the inhibitor.

As used herein, the term “candidate substance” refers to any molecule that may potentially decrease, reduce or inhibit PKCδ activity or function. Candidate compounds may include fragments or parts of naturally-occurring compounds or may be found as active combinations of known compounds which are otherwise inactive. The candidate substance can be a nucleic acid, a polypeptide, a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Clemons et al., 2001; Blackwell et al., 2001). Chemical genetics is the technology that uses small molecules to modulate the functions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The most useful pharmacological compounds may be compounds that are structurally related to compounds which interact naturally with compounds that modulate PKCδ activity. Creating and examining the action of such molecules is known as “rational drug design,” and include making predictions relating to the structure of target molecules. Thus, it is understood that the candidate substance identified by the present invention may be a small molecule inhibitor or any other compound (e.g., polypeptide or polynucleotide) that may be designed through rational drug design starting from known inhibitors of PKCδ.

The goal of rational drug design is to produce or manufacture structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule similar to PKCδ, and then design a molecule for its ability to interact with an PKC6-related molecule. This could be accomplished by X-ray crystallography, computer modeling or by a combination of both approaches. The same approach may be applied to identifying interacting molecules of PKCδ.

It also is possible to use antibodies to ascertain the structure of a target compound or activator. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

B. In Vitro Assays

A quick, inexpensive and easy assay to run is a binding assay. Binding of a molecule to a target (e.g., PKCδ) may, in and of itself, be agonist, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to PKCδ molecules or fragments thereof are provided.

A target PKCδ protein may be either free in solution, fixed to a support, expressed in or on the surface of a cell. Either the PKCδ protein or the compound may be labeled, thereby indicating if binding has occurred. In another embodiment, the assay may measure the binding of PKCδ to a natural or artificial substrate or binding partner. Competitive binding assays can be performed in which one of the agents is labeled. Usually, the target PKCδ protein will be the labeled species, decreasing the chance that the labeling will interfere with the binding moiety's function. One may measure the amount of free label versus bound label to determine binding or activation of binding. These approaches may be utilized on PKCδ molecules.

A technique for high throughput screening of compounds is described in WO 84/03564. Large numbers of small peptide test compounds are synthesized on a solid substrate, such as plastic pins or some other surface. The peptide test compounds are reacted with, for example, PKCδ protein and washed. Bound polypeptide is detected by various methods.

C. In Cyto Assays

Various cell lines that express PKCδ related proteins can be utilized for screening of candidate substances. For example, cells containing PKCδ proteins with an engineered indicator can be used to study various functional attributes of candidate compounds. In such assays, the compound would be formulated appropriately, given its biochemical nature, and contacted with a target cell. This same approach may utilized to study various functional attributes of candidate compounds that effect PKCδ.

D. In Vivo Assays

The present invention particularly contemplates the use of various animal models. Treatment of animals with test compounds (e.g., PKCδ inhibitors) involve the administration of the compound, in an appropriate form, to the animal. Administration is by any route that could be utilized for clinical or non-clinical purposes. Specifically contemplated are ophthalmic administration, for example, it is contemplated that all local routes to the eye may be used, including topical, subconjunctival, periocular, retrobulbar, subtenon, intracameral, intravitreal, intraocular, subretinal, posterior juxtascleral, and suprachoroidal administration.

E. Production of an Inhibitor

In an extension of any of the previously described screening assays, the present invention also provide for methods of producing or manufacturing PKCδ inhibitors. The methods comprising any of the preceding screening steps followed by an additional step of “producing or manufacturing the candidate substance identified as an PKCδ inhibitor” the screened activity. Manufacturing can entail any well known and standard technique used by those of skill in the art, such as synthesizing the compound and/or deriving the compound from a natural source.

### V. Pharmaceutics and Formulations

A. Dosage

The phrase “pharmaceutically effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to decrease, reduce and/or inhibit the enzyme activity of protein kinase C-delta (PKCδ). The effective amount may vary depending on such factors as the disease or condition being treated, the particular composition being administered, or the severity of the disease or condition. One of skill in the art would be familiar with determining an effective amount of a particular agent without necessitating undue experimentation.

The phrase “pharmaceutically acceptable” is art-recognized and refers to compositions, polymers and other materials and/or dosage forms which are suitable for use in contact with the tissues of human beings and animals without excessive toxicity, irritation, allergic response, or other problem or complication, commensurate with a reasonable benefit/risk ratio as determined by one of ordinary skill in the art.

The amount of agent or compound to be included in the compositions or applied in the methods set forth herein will be whatever amount is pharmaceutically effective and will depend upon a number of factors, including the identity and potency of the chosen agent or compound. One of ordinary skill in the art would be familiar with factors that are involved in determining a pharmaceutically effective dose of an agent or compound.

In particular embodiments, the composition is administered once a day. However, the compositions of the present invention may also be formulated for administration at any frequency of administration, including once a week, once every 5 day, once every 3 days, once every 2 days, twice a day, three times a day, four times a day, five times a day, six times a day, eight times a day, every hour, or any greater frequency. One of ordinary skill in the art would be familiar with establishing a therapeutic regimen. Factors involved in this determination include the disease to be treated, particular characteristics of the subject.

B. Formulations

Regarding the methods set forth herein, a PKCδ inhibitor composition can be formulated in any manner known to those of ordinary skill in the art. In the compositions set forth herein, the concentration of a PKCδ inhibitor can be any concentration known or suspected by those of ordinary skill in the art to be of benefit in the treatment and/or prevention of glaucoma, elevated intraocular pressure or ocular hypertension.

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain non-limiting embodiments, the pharmaceutical compositions may comprise, for example, at least about 0.1%, by weight or volume, of an active ingredient. In other embodiments, the active ingredient may comprise between about 2% to about 75% of the weight or volume of the unit, or between about 25% to about 60%, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein.

In certain embodiments of the present invention, the compositions set forth herein can include more than one PKCδ inhibitors. One of ordinary skill in the art would be familiar with preparing and administering pharmaceutical compositions that include more than one therapeutic agent. In some embodiments, the composition includes one or more additional therapeutic agents that are not PKCδ inhibitors.

In addition to the PKCδ inhibitors, the compositions of the present invention optionally comprise one or more excipients. Excipients commonly used in pharmaceutical compositions include, but are not limited to, carriers, tonicity agents, preservatives, chelating agents, buffering agents, surfactants and antioxidants.

A person of ordinary skill will recognize that the compositions of the present invention can include any number of combinations of ingredients (e.g., active agent, polymers, excipients, etc.). It is also contemplated that that the concentrations of these ingredients can vary. For example, in one-non-limiting aspect, a composition of the present invention can include at least about 0.0001% to about 0.001%, 0.001% to about 0.01%, 0.01% to about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient.

Any of a variety of carriers may be used in the formulations of the present invention including water, mixtures of water and water-miscible solvents, such as C₁-C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, mixtures of those polymers. The concentration of the carrier is, typically, from 1 to 100000 times the concentration of the active ingredient.

Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1 and the like. Suitable chelating agents include sodium edetate and the like. Suitable buffering agents include phosphates, borates, citrates, acetates and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable antioxidants include sulfites, ascorbates, BHA and BHT. The compositions of the present invention optionally comprise an additional active agent.

In particular embodiments, the compositions are suitable for application to mammalian eyes. For example, for ophthalmic administration, the formulation may be a solution, a suspension, a gel, or an ointment.

In preferred aspects, the compositions that includes PKCδ inhibitors will be formulated for topical application to the eye in aqueous solution in the form of drops. The term “aqueous” typically denotes an aqueous composition wherein the carrier is to an extent of >50%, more preferably >75% and in particular >90% by weight water. These drops may be delivered from a single dose ampoule which may preferably be sterile and thus rendering bacteriostatic components of the formulation unnecessary. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

The compositions of the present invention are preferably not formulated as solutions that undergo a phase transition to a gel upon administration to the eye.

In addition to the one or more PKCδ inhibitors, the compositions of the present invention may contain other ingredients as excipients. For example, the compositions may include one or more pharmaceutically acceptable buffering agents, preservatives (including preservative adjuncts), non-ionic tonicity-adjusting agents, surfactants, solubilizing agents, stabilizing agents, comfort-enhancing agents, polymers, emollients, pH-adjusting agents and/or lubricants.

For topical formulations to the eye, the formulation are preferably isotonic, or slightly hypotonic in order to combat any hypertonicity of tears caused by evaporation and/or disease. The compositions of the present invention generally have an osmolality in the range of 220-320 mOsm/kg, and preferably have an osmolality in the range of 235-260 mOsm/kg. The compositions of the invention have a pH in the range of 5-9, preferably 6.5-7.5, and most preferably 6.9-7.4.

The formulations set forth herein may comprise one or more preservatives. Examples of preservatives include quaternary ammonium compounds, such as benzalkonium chloride or benzoxonium chloride. Other examples of preservatives include alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, or sorbic acid.

In certain embodiments, the PKCδ inhibitors are formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. The formulation of the present invention may be used with contact lenses or other ophthalmic products.

In some embodiments, the compositions set forth herein have a viscosity of 0.5-10 cps, preferably 0.5-5 cps, and most preferably 1-2 cps. This relatively low viscosity insures that the product is comfortable, does not cause blurring, and is easily processed during manufacturing, transfer and filling operations.

C. Route of Administration

The PKCδ inhibitor compositions for use in the methods of the invention may be administered via any viable delivery method or route, however, local administration is preferred. It is contemplated that all local routes to the eye may be used including topical, subconjunctival, periocular, retrobulbar, subtenon, intracameral, intravitreal, intraocular, subretinal, juxtascleral and suprachoroidal administration. Systemic or parenteral administration may be feasible including but not limited to intravenous, subcutaneous, and oral delivery. The most preferred method of administration will be intravitreal or subtenon injection of solutions or suspensions, or intravitreal or subtenon placement of bioerodible or non-bioerodible devices, or by topical ocular administration of solutions or suspensions, or posterior juxtascleral administration of a gel formulation.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Ocular Safety Evaluation in New Zealand Albino Rabbits

The ability of certain PKCδ inhibitors to safely lower IOP may be evaluated in certain embodiments by means of in vivo assays using New Zealand albino rabbits and/or Cynomolgus monkeys.

For example, both eyes of New Zealand albino rabbits are topically dosed with one 30 μL aliquot of a test compound in a vehicle. Animals are monitored continuously for 0.5 hr post-dose and then every 0.5 hours through 2 hours or until effects are no longer evident.

### Example 2

### Acute IOP Response in New Zealand Albino Rabbits

Intraocular pressure (IOP) is determined with a Mentor Classic 30 pneumatonometer after light corneal anesthesia with 0.1% proparacaine. Eyes are rinsed with one or two drops of saline after each measurement. After a baseline IOP measurement, test compound is instilled in one 30 μL aliquot to one or both eye of each animal or compound to one eye and vehicle to the contralateral eye. Subsequent IOP measurements are taken at 0.5, 1, 2, 3, 4, and 5 hours.

### Example 3

### Acute IOP Response in Cynomolgus Monkeys

Intraocular pressure (IOP) is determined with an Alcon pneumatonometer after light corneal anesthesia with 0.1% proparacaine as previously described (Sharif et al., 2001; May et al., 2003). Eyes are rinsed with one or two drops of saline after each measurement. After a baseline IOP measurement, test compound is instilled in one or two 30 μL aliquots to the selected eyes of cynomolgus monkeys. Subsequent IOP measurements are taken at 1, 3, and 6 hours. Right eyes of all animals had undergone laser trabeculoplasty to induce ocular hypertension. All left eyes are normal and thus have normal IOP.

### Example 4

### Measurement of TGFβ2 Mediated Responses

Cultured transformed human TM cells (“GTM-3”; see Pang I H, et al., 1994) were grown in a growth medium consisting of Dulbecco's modified Eagle's medium with Glutamax I (Gibco/Invitrogen, Grand Island, N.Y.) supplemented with 10% fetal bovine serum (Hyclone, Logan, Utah) and 50 μg/mL gentamicin (Gibco/Invitrogen). For assay, cultures were enzymatically-dissociated (TrypLE Express; Gibco/Invitrogen) then seeded into 24-well plates (Corning Costar, Acton, Mass.) and allowed to grow until monolayers reached approximately 90-95% confluence. Culture medium was then replaced with 0.25 mL serum- and antibiotic-free medium containing the appropriate test compound(s). Cells were incubated 24 h, at 5% CO₂ and 37° C. Aliquots of culture supernatants were then assayed using ELISA kits for fibronectin (AssayPro, St. Charles, Mo.), PAI-I (American Diagnostica, Stamford, Conn.) and procollagen Type I C-peptide (TaKaRa Bio, Shiga, Japan).

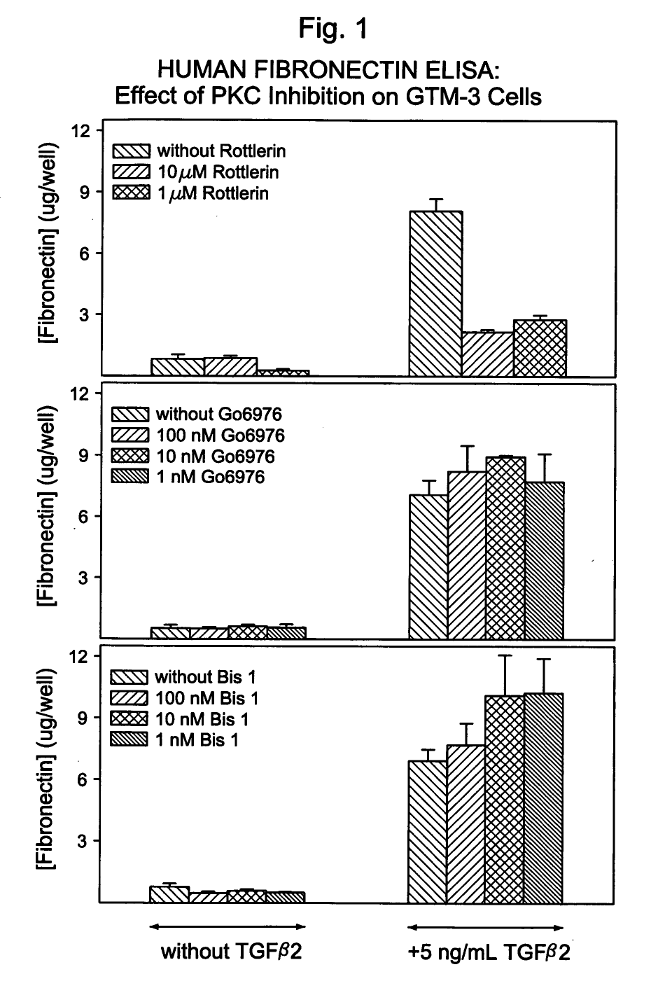

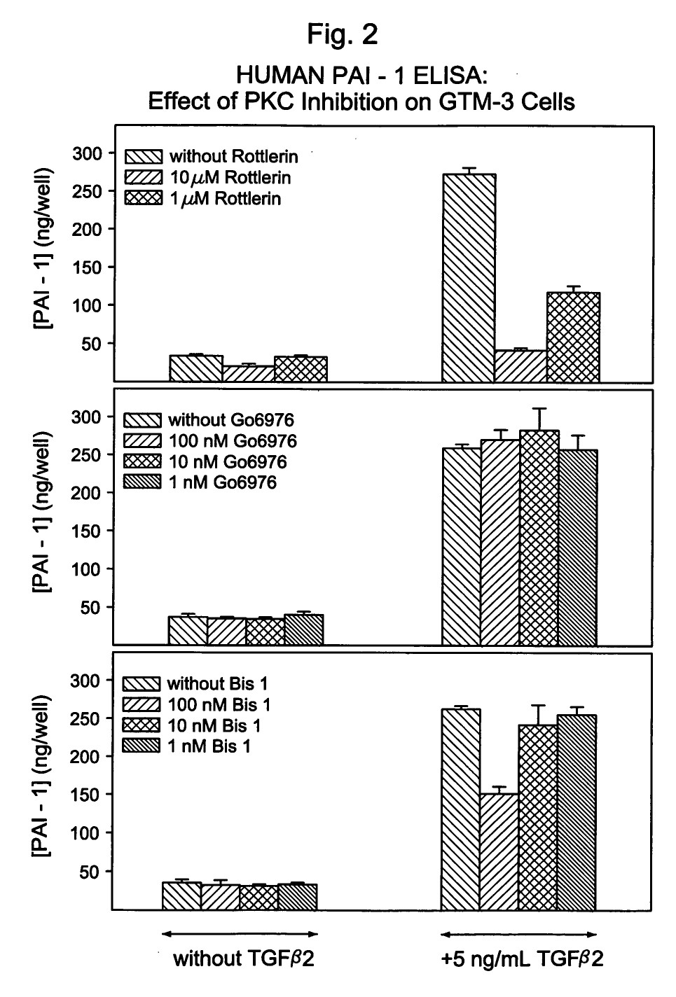

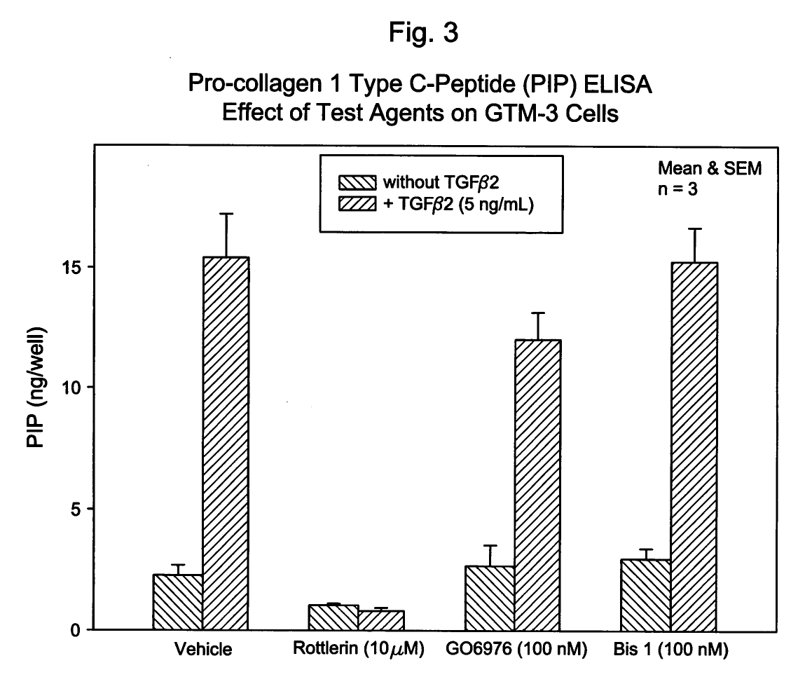

The test compounds used were rottlerin (a specific PKC-delta inhibitor), Gö6976 (inhibits the calcium-dependent PKC isoforms alpha, beta, & mu) and bisindolylmaleimide I (a broad-spectrum PKC inhibitor). FIG. 1 shows the effect of these test compounds on fibronectin synthesis. FIG. 2 shows the effect of these test compounds on PAI-1. FIG. 3 shows the effect of these test compounds on PIP. These results indicate that rottlerin was efficient at inhibiting PKCδ resulting in decreases in TGFβ2-induced mediated responses.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
1. WO 84/03564
2. Bennett et al., J of Molecular Recognition, Vol. 8:52-58, 1995
3. Blackwell et al., Chem. Biol., Vol. 8(12):1167-82, 2001
4. Clemons et al., Chem. Biol., Vol. 8(12):1183-95, 2001
5. Jinnin et al., 2005
6. Johanson et al., J. of Biological Chem, Vol. 270(16):9459-9471, 1995
7. Kandabashi et al., Arterioscler Thromb Vasc Biol, Vol. 23:2209-2214, 2003
8. May et al., J. Pharmacol. Exp. Ther., Vol. 306:301-309, 2003
9. Mueller et al., Exp Clin Endocrinol Diabetes, Vol. 105:32-38, 1997
10. Pang et al., Curr Eye Res., Vol. 13:51-63, 1994
11. Rao et al., Mol. Vis., Vol. 11:288-297, 2005
12. Runyan et al., Am J Physiol Renal Physiol, Vol. 285:F413-F422, 2003
13. Sharif et al., J. Ocular Pharmacol. Ther., Vol. 17:305-317, 2001
14. Zhang et al., J Biol Chem, Vol. 27:28315-28319, 2004

What is claimed is:

1. A method of decreasing intraocular pressure in a subject comprising administering to the subject an effective amount of a composition comprising a protein kinase C-delta (PKCδ) inhibitor.

2. The method of claim 1, wherein the subject is at risk of developing glaucoma.

3. The method of claim 1, wherein the subject has elevated intraocular pressure in at least one eye.

4. The method of claim 3, wherein the intraocular pressure in at least one eye is equal to or greater than 21 mm Hg.

5. The method of claim 1, wherein the subject has glaucoma.

6. The method of claim 5, wherein the glaucoma is primary open angle glaucoma.

7. The method of claim 1, wherein the PKCδ inhibitor is selected from the group consisting of bisindolylmaleimide I, bisindolylmaleimide II, bisindolylmaleimide III, bisindolylmaleimide IV, calphostin C, chelerythrine chloride, ellagic acid, Gö 7874, Gö 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, D-erythro-sphingosine and combinations thereof.

8. The method of claim 5, wherein the PKCδ inhibitor is rottlerin.

9. The method of claim 1 further comprising administering a compound selected from the group consisting of a β-blocker, a prostaglandin analog, a carbonic anhydrase inhibitor, an α2 agonist, a miotic, a neuroprotectant, a rho kinase inhibitor, and a combination thereof.

10. A method of treating and/or preventing glaucoma comprising administering to a subject having glaucoma or a subject susceptible to having glaucoma an effective amount of a composition comprising a protein kinase C-delta inhibitor (PKCδ).

11. The method of claim 10, wherein the subject has elevated intraocular pressure.

12. The method of claim 11, wherein the intraocular pressure is at least 21 mm Hg in at least one eye of the subject.

13. The method of claim 10, wherein the subject has an intraocular pressure less than 21 mm Hg in at least one eye of the subject.

14. The method of claim 10, wherein the PKCδ inhibitor is selected from the group consisting of bisindolylmaleimide I, bisindolylmaleimide II, bisindolylmaleimide III, bisindolylmaleimide IV, calphostin C, chelerythrine chloride, ellagic acid, Gö 7874, Gö 6983, H-7, Iso-H-7, hypericin, K-252a, K-252b, K-252c, melittin, NGIC-I, phloretin, staurosporine, polymyxin B sulfate, protein kinase C inhibitor peptide 19-31, protein kinase C inhibitor peptide 19-36, protein kinase C inhibitor (EGF-R Fragment 651-658, myristoylated), Ro-31-8220, Ro-32-0432, rottlerin, safingol, sangivamycin, D-erythro-sphingosine and combinations thereof.

15. The method of claim 14, wherein the PKCδ inhibitor is rottlerin.

16. A method of manufacturing a protein kinase C-delta (PKCδ) inhibitor comprising:
(a) providing a candidate substance suspected of decreasing PKCδ activity;
(b) selecting the PKCδ inhibitor by assessing the ability of the candidate substance to decrease PKCδ; and
(c) manufacturing the selected PKCδ inhibitor.

17. The method of claim 16, wherein the candidate substance is a small molecule.

18. A method of treating and/or preventing increased intraocular pressure comprising administering to a subject an effective amount of a PKCδ inhibitor identified in claim 16.

19. A method of treating and/or preventing glaucoma comprising administering to a subject an effective amount of PKCδ inhibitor identified in claim 16.
